(19) United States
(12) Patent Application Publication
(10) Pub. No.: US 2009/0048633 A1
(43) Pub. Date: Feb. 19, 2009

(54) Fixture

(76) Inventors: Tae-Gwan Eom, Busan (KR); Byung-Kook Kim, Busan (KR)

Correspondence Address: RABIN & Berdo, PC, 1101 14TH STREET, NW, SUITE 500, WASHINGTON, DC, 20005, US

(21) Appl. No.: 10/583,150

(22) PCT Filed: Jan. 27, 2006
(86) PCT No.: PCT/KR06/00316

§ 371 Date: Jun. 16, 2006

(51) Int. Cl. A61B 17/04; A61B 17/58

(52) U.S. Cl. 606/301; 606/60

(57) ABSTRACT

A screw type fixture is disclosed. In the fixture of the present invention, small screw threads (231) are formed on a ridge of a large screw thread (233) formed on the circumferential outer surface of the fixture, so that reliable fixing force of the fixture to cancellous bone is ensured by the increased contact surface, and stress around the large screw thread (233) is distributed. Furthermore, in the present invention, the pitch of the screw thread of a cortical bone coupling part (33) of the fixture, which is implanted in cortical bone, and the difference between the outer diameter thereof and the root diameter thereof are less than those of a cancellous bone coupling part (34) of the fixture, so that stress otherwise concentrated on the cortical bone is distributed to the cancellous bone, thus preventing bone resorption, and promoting osseointegration.

### TECHNICAL FIELD

The present invention relates, in general, to screw type fixtures and, more particularly, to a fixture, in which small screw threads are formed on a ridge of a large screw thread formed on a circumferential outer surface of the fixture, so that reliable fixing force of the fixture to a cancellous bone is ensured by the increased contact surface area, and stress concentrated on the large screw thread is distributed, and in which a pitch of the screw thread of a portion of the fixture that is positioned in a cortical bone, and the difference between the outer diameter thereof and the root diameter thereof are less than those of a portion of the fixture that is positioned in a cancellous bone, so that stress otherwise concentrated on the cortical bone is distributed to the cancellous bone, thus preventing bone resorption and promoting osseointegration.

### BACKGROUND ART

Generally, screw type fixtures have screw threads on circumferential outer surfaces of the main bodies thereof and are used as support members for fastening dental or orthopedic prosthetic appliances to bones. Such a screw type fixture is inserted into an insertion hole in bone tissue which is formed using a drill or the like at a desired position, at which an implant is to be implanted in the bone tissue. The insertion hole of the bone tissue is formed to have a diameter slightly less than the diameter of the fixture. Furthermore, a cutting edge is formed on the fixture, so that, when the fixture is tightened into the insertion hole of the bone tissue, an internal thread is formed by the cutting edge on the circumferential inner surface of the insertion hole.

As shown in FIG. 2, the bone tissue consists of a cortical bone 81 and a cancellous bone 83. The cancellous bone is relatively soft bone tissue in the middle of bone. The cortical bone 81 is harder than the cancellous bone and is a relatively thin layer surrounding the cancellous bone. Therefore, when the fixture is implanted in the bone, the contact length of the fixture with the cancellous bone is greater than the contact length thereof with the cortical bone.

However, the conventional fixture has several problems. One of them, a bone resorption phenomenon, is a major problem occurring with the conventional fixture. The term “bone resorption” means a phenomenon in which bone tissue around the fixture is degenerated as the amount thereof is reduced. The bone resorption-phenomenon reduces the fixing force of the fixture and thus decreases the stability of the implant and causes damage to the implant. Particularly, in the case of the dental implant, bone resorption may entail the deposition of dental calculus, which causes inflammation of the gum tissue surrounding the fixture, or may cause the gum tissue to grow in an undesired direction, that is, downwards, along the end of the fixture which is exposed outside. As such, bone resorption decreases the stability of the implant and, in addition, deteriorates the appearance thereof. Therefore, bone resorption must be prevented from occurring.

The biological cause of bone resorption has not been clearly disclosed yet. It has merely been recognized that high stimulation and/or low stimulation due to unevenly distributed stress applied to the bone tissue around the fixture, that is, due to stress concentration, accelerates bone resorption. Therefore, to prevent bone resorption and to promote osseointegration between the fixture and the bone tissue, it is necessary to evenly distribute stress.

In the stress concentration causing the bone resorption problem of the fixture, there is partial stress concentration occurring at each ridge of the screw thread and overall stress concentration caused by the imbalance of stress throughout the entire fixture. As shown in FIG. 8, partial stress concentration mainly occurs around a sharp part of the s crew thread, such as a ridge. This partial stress concentration causes resorption of bone tissues around each ridge of the screw thread. The overall stress concentration results from uneven distribution of stress around portions of the fixture implanted both in the cortical bone and in the cancellous bone, which have different strength from each other. As shown in FIG. 2, when the fixture is implanted in the bone, a relatively high stress concentration occurs around the cortical bone, which is the stiffest part of the bone, so that bone resorption is mainly caused around the cortical bone.

As shown in FIG. 1, the conventional fixture includes a head part 13, to which a prosthetic appliance (not shown) is mounted, a threaded part 11, which is implanted in a bone, and a cutting edge 15, which conducts a self-tapping function. The threaded part 11 is formed on a circumferential outer surface of a main body in a triangular thread or trapezoidal thread shape. Typically, a single screw thread 111 is formed throughout the entire length of the main body. Because the screw thread 111 is disposed in relatively soft cancellous bone, there is a general tendency to relatively increase both the pitch p of the screw thread 111 and the difference between an outer diameter d1 and a root diameter d2 of the screw thread 111 to increase the fixing force of the fixture, as shown in FIG. 1.

As the pitch p of the screw thread 111 and the difference between an outer diameter d1 and a root diameter d2 of the screw thread 111 are increased, the fixing force of the fixture is increased, but partial stress concentration owing to the screw thread is also increased.

Therefore, it is required to solve the problem of partial stress concentration of the screw thread of the fixture while maintaining the initial fixing force of the fixture.

Furthermore, in the conventional fixture 1, the triangular screw thread or the trapezoidal screw thread is formed throughout the entire threaded part 11 in a single shape. This induces an overall stress imbalance, in which stress is relatively concentrated around the cortical bone 81, which is stiffer than the cancellous bone 83. Due to the overall stress imbalance of the fixture, bone resorption is mainly caused around the cortical bone. This bone resorption in the cortical bone reduces the stability of the prosthetic appliance and deteriorates the appearance thereof. Therefore, to prevent bone resorption around the cortical bone from occurring the necessity to distribute stress, concentrated in the cortical bone, to the cancellous bone, has arisen.

Furthermore, although the triangular screw thread or the trapezoidal screw thread of the conventional fixture 1 increases the contact surface between the fixture and the bone tissue, compared to a cylindrical shape, the contact surface should be further increased to ensure the stability of the prosthetic appliance.

### DISCLOSURE OF INVENTION

### Technical Solution

Accordingly, the present invention has been made keeping in mind the above problems occurring in the prior art, and an object of the present invention is to provide a fixture which prevents stress from being unevenly applied to a screw thread thereof and maintains the initial fixing force of the fixture to a cancellous bone, thus preventing bone resorption and promoting osseointegration.

Another object of the present invention is to provide a fixture in which coupling force thereof with respect to bone tissue is enhanced by an increased contact surface area between the fixture and the bone tissue.

A further object of the present invention is to provide a fixture in which stress concentrated on the cortical bone is distributed to the cancellous bone, so that the stress is evenly distributed to the entire fixture, thus preventing bone resorption and promoting osseointegration.

In order to accomplish the above objects, the present invention has the following construction.

According to a first embodiment, the present invention provides a screw type fixture to be implanted in bone tissue. The fixture includes an uppermost part protruding outside the bone tissue when implanted in the bone tissue, and a body part placed in the bone tissue. The body part has a cortical bone coupling part installed in cortical bone, and a cancellous bone coupling part installed in cancellous bone. The cancellous bone coupling part has a large cancellous screw thread formed on a circumferential outer surface of the cancellous bone coupling part, and a small cancellous screw thread formed on a ridge of the large cancellous screw thread. The cortical bone coupling part has a small cortical screw thread, which has a pitch, a root diameter and an outer diameter almost equal to a pitch, a root diameter and an outer diameter of the small cancellous screw thread.

According to a second embodiment, the present invention provides a screw type fixture to be implanted in bone tissue. The fixture includes an uppermost part protruding outside the bone tissue when implanted in the bone tissue and a body part placed in the bone tissue. The body part has a cortical bone coupling part installed in cortical bone, and a cancellous bone coupling part installed in cancellous bone. The cancellous bone coupling part includes a large cancellous screw thread formed on a circumferential outer surface of the cancellous bone coupling part, and a small cancellous screw thread formed on a ridge of the large cancellous screw thread. The cortical bone coupling part includes a large cortical screw thread extending from the large cancellous screw thread, and a small cortical screw thread formed on a ridge of the large cortical screw thread. The number of small cortical screw threads is greater than the number of small cancellous screw threads.

In the fixture according to a third embodiment of the present invention, the large cortical screw thread may have a lead equal to that of the large cancellous screw thread.

In the fixture according to a fourth embodiment of the present invention, a root diameter of the large cortical screw thread may be increased from a lower end thereof to an upper end thereof and be equal to a root diameter of the small cortical screw thread at the upper end thereof.

In the fixture according to a fifth embodiment of the present invention, a root diameter of the large cancellous screw thread may be constant throughout the cancerous bone coupling part.

According to a sixth embodiment of the present invention, the fixture may further include a plurality of longitudinal grooves formed in a circumferential outer surface of the cortical bone coupling part.

### BRIEF DESCRIPTION OF THE DRAWINGS

FIG. 1 is a front view showing a conventional fixture

FIG. 2 is a sectional view showing the fixture of FIG. 1 implanted in bone tissue

FIG. 3 is a front view of a fixture, according to an embodiment of the present invention;

FIG. 4 is a sectional view of the fixture of FIG. 3;

FIG. 5 is a front view of a fixture, according to another embodiment of the present invention;

FIG. 6 is a front view of a fixture, according to a further embodiment of the present invention;

FIG. 7 is a front view of a fixture, according to a still another embodiment of the present invention;

FIG. 8 is a schematic view showing a process of implanting the fixture into bone tissue according to the present invention;

FIG. 9 is a schematic view showing the fixture implanted in the bone tissue according to the present invention;

FIG. 10 is a stress distribution view showing an FEM analysis of the conventional fixture; and

FIG. 11 is a stress distribution view showing an FEM analysis of the fixture according to the present invention.

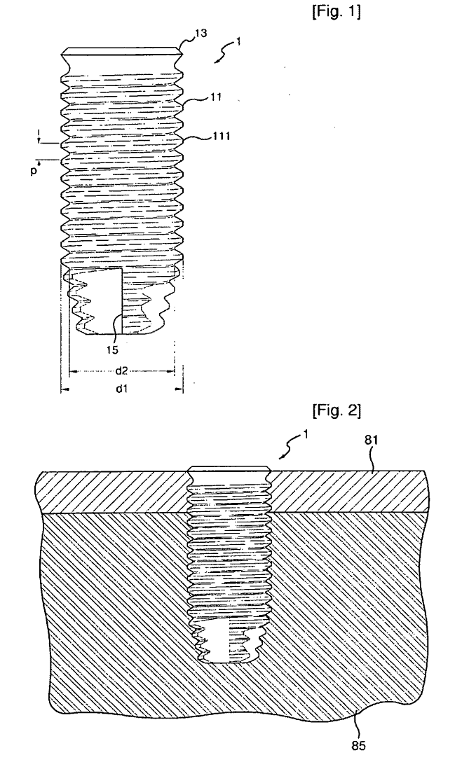

### BEST MODE FOR CARRYING OUT THE INVENTION

Hereinafter, preferred embodiments of the present invention will be described in detail with reference to the attached drawings.

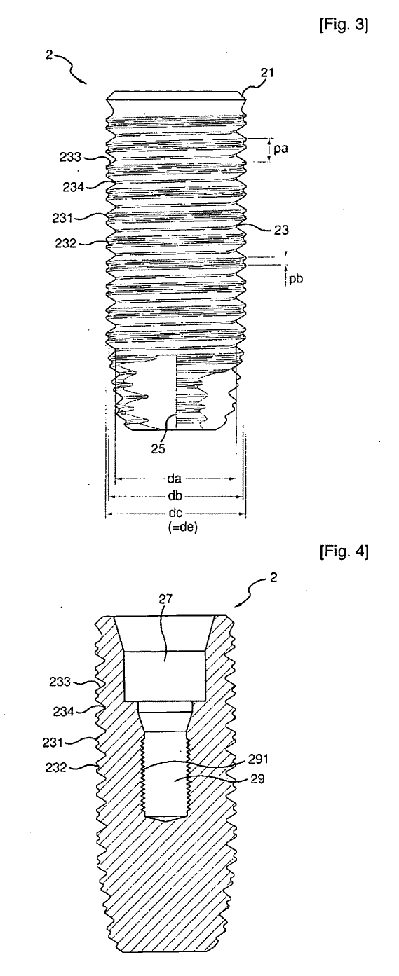

FIG. 3 is a front view of a fixture 2, according to an embodiment of the present invention. FIG. 4 is a longitudinal sectional view of the fixture 2 of FIG. 3. The fixture 2 of FIG. 3 is a kind of internal type dental fixtures to be implanted in alveolar bone for holding dental prostheses. The fixture 2 has a cylindrical shape and is made of bio-friendly metal, such as pure titanium or titanium alloy to promote osseointegration between bone tissue and the fixture. The fixture 2 includes an uppermost part 21, which is exposed outside the bone tissue when the fixture 2 is implanted in the bone tissue, a body part 23, which is provided under the uppermost part and placed in the bone tissue, and a cutting edge 25, which is formed on a lower end of the body part for self-tapping. An adaptor seating hole 27, into which an adaptor (not shown) is inserted, and a screw locking hole 29, into which a screw (not shown) is tightened, are formed in the fixture 2.

The uppermost part 21 is positioned at the uppermost end of the fixture 2 and has a conical shape which is reduced in diameter from the bottom to the top. Only the uppermost part 21 protrudes outside the bone tissue when the fixture is implanted in the bone tissue. It is preferable that the uppermost part 21 be much shorter than the overall length of the fixture.

The body part 23 is placed in the bone tissue when the fixture is implanted in the bone tissue. An external thread is formed on a circumferential outer surface of the body part 23. The external thread is formed in the same shape from the lower end of the body part 23 to the uppermost part 21. The external thread consists of a large screw thread 233, a large thread valley 234, small screw threads 231 and a small thread valley 232.

The large screw thread 233 has a trapezoidal shape and is formed along the outer surface of the body part 23 in a single screw thread or double screw thread arrangement. The large screw thread 233 has an outer diameter of dc, a root diameter of da and a pitch of Pa. Since the large screw thread 233 and the large thread valley 232 serve to fasten the fixture 2 to the alveolar bone, the pitch Pa of the large screw thread 233 and the difference between the outer diameter dc and the root diameter da should be relatively great. Preferably, in consideration of fixing ability of the fixture and workability in machining the small screw threads 231 and the small thread valley 232, the pitch Pa of the large screw thread 233 is 0.8 mm. The several small screw threads 231 and the small thread valley 232 are formed in the ridge of the large screw thread 233.

As shown in FIG. 4, each small screw thread 231, which is formed in the ridge of the large screw thread 233, has an outer diameter of de, a root diameter of db and a pitch of Pb. The small screw threads 231 decrease partial stress concentration around the large screw thread 233, thus preventing bone resorption. Furthermore, due to the small screw threads 231, the contact surface area between the fixture 2 and the bone tissue is increased, thereby the fixing ability of the fixture 2 is increased. Preferably, the root diameter dd of the small screw thread 231 is greater than the root diameter da of the large screw thread 233, and the outer diameter de of the small screw thread 231 is equal to the outer diameter dc of the large screw thread 233. According to the test, the surface area of the fixture 2 of the present invention, in which two small screw threads 231 are formed in the large screw thread 233, was increased by 7% over that of the conventional fixture having only a large screw thread 233 with a pitch of 0.8 mm. In FIGS. 3a and 3b, although two small screw threads 231 and a single small thread valley 232 are formed in the fixture 2, the number of small screw threads 231 may be changed according to the intended purpose.

The cutting edge 25 is defined by axially cutting part of the screw thread formed on the lower end of the body part 23. The cutting edge 25 conducts a self-tapping function. That is, after an insertion hole, having a diameter slightly less than that of the body part 23, is formed in the bone tissue at a desired position, when the fixture is tightened into the insertion hole of the bone tissue, the cutting edge 35 forms an internal thread on the circumferential inner surface of the insertion hole.

As shown in FIG. 4, the adaptor seating hole 27 has a cylindrical shape and is formed in the body part 23 from the uppermost part 21 to a predetermined depth. The screw locking hole 29 axially extends from the lower end of the adaptor seating hole 27. An internal thread 291 is formed on the circumferential inner surface of the screw locking hole 29, so that the screw (not shown) engages with the internal thread 291 when it is inserted into the screw locking hole 29.

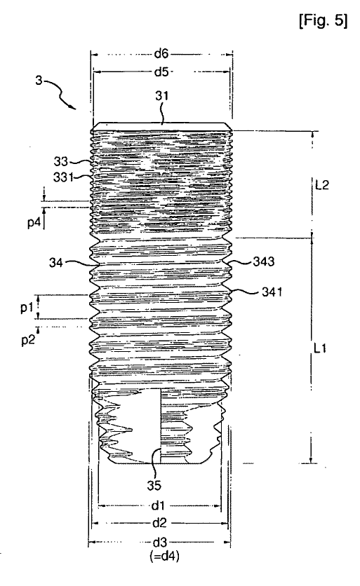

Meanwhile, as shown in FIG. 5, a fixture 3 according to another embodiment of the present invention includes an uppermost part 31, a cortical bone coupling part 33, a cancellous bone coupling part 34, a cutting edge 35, an adaptor seating hole and a screw locking hole. The general shape and material of the fixture 3 of this embodiment are equal to those of the fixture 2 according to the embodiment shown in FIGS. 3 and 4. Furthermore, the constructions and functions of the uppermost part 31, the cutting edge 35, the adaptor seating bole and the screw locking hole are equal to the uppermost part 21, the cutting edge 25, the adaptor seating hole 27 and the screw locking hole 29 of the fixture 2 of the embodiment of FIGS. 3 and 4, therefore only the cortical bone coupling part 33 and the cancellous bone coupling part 34 will be explained herein below.

When the fixture 3 is implanted in the bone tissue, most of the cancellous bone coupling part 34 is placed in the cancellous bone. The cancellous bone coupling part 34 is formed on the lower portion of the fixture 3 and has a length of L1. A large cancellous screw thread 343 and small cancellous screw threads 341 are formed on the circumferential outer surface of the cancellous bone coupling part 34.

The large cancellous screw thread 343 is formed throughout the entire circumferential outer surface of the cancellous bone coupling part 34 and has an outer diameter of d3, a root diameter of d1 and a pitch of p1. The small cancellous screw threads 341 are formed on a ridge of the large cancellous screw thread 343 and have outer diameters of d4, a root diameter of d2 and a pitch of p2. Of course, the number of small cancellous screw threads 341 may be changed as required. The constructions and functions of the large cancellous screw thread 343 and the small cancellous screw thread 341 are equal to those of the large screw thread 233 and the small screw thread 231 of the embodiment shown in FIGS. 3a and 3b.

Meanwhile, most of the cortical bone coupling part 33 is positioned in the cortical bone when the fixture 3 is implanted in the bone tissue. The cortical bone coupling part 33 has a predetermined length L2 between the uppermost part 31 and the cancellous bone coupling part 34. The cortical bones of the bone tissues differ in thickness for each person and, typically, they are not thicker than 3 mm. Therefore, it is preferable that the length L2 of the cortical bone coupling part 33 be 3.5 mm or less so as to provide some extra length. Furthermore, a small cortical screw thread 331 is formed on the circumferential outer surface of the cortical bone coupling part 33.

The small cortical screw thread 331 is formed throughout the entire length of the cortical bone coupling part 33 and has an outer diameter of d6, a root diameter of d5 and a pitch of p4. It is preferable that the pitch p4 of the small cortical screw thread 331 be ¼ of the pitch p1 of the large cancellous screw thread 343, that is, it is equal to the pitch p2 of the small cancellous screw thread 341. Preferably, the number of small cortical screw threads 331 is 4 times the number of large cancellous screw threads 343, so that the lead of the small cortical screw thread 331 is equal to that of the small cancellous screw thread 341. Furthermore, the root diameter d5 and the outer diameter d6 of the small cortical screw thread 331 are respectively equal to the root diameter d2 and the outer diameter d4 of the small cancellous screw thread 341, so that the fixture 3 can be implanted into the bone tissue without the small cortical screw thread 331 damaging the internal thread of the bone tissue, which has been formed by the small cancerous screw thread 341.

Because the pitch p4 and the difference between the outer diameter d6 and the root diameter d5 of the cortical screw thread 331 are less than those of the large cancellous screw thread 343, stress applied to the cortical bone coupling part 33 is reduced, and so the stress is distributed to the cancellous bone coupling part 34. Hence, thanks to the small cortical screw thread 331, stress concentration relative to the cortical bone is distributed to the cancellous bone, thus preventing resorption of the cortical bone.

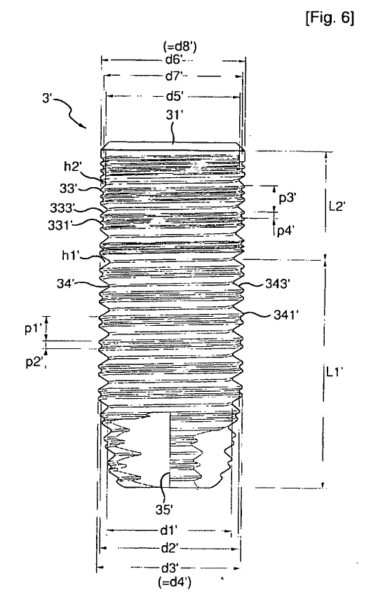

As shown in FIG. 6, a fixture 3′ according to a further embodiment of the present invention includes an uppermost part 31′, a cortical bone coupling part 33′, a cancellous bone coupling part 34′, a cutting edge 35′, an adaptor seating bole and a screw locking hole. The cancellous bone coupling part 34′ has a large cancellous screw thread 343′ on a circumferential outer surface thereof and small cancellous screw threads 341′, which are formed on the ridge of the large cancellous screw thread 343′. The cortical bone coupling part 33′ has small cortical screw threads 331′ and a large cortical screw thread 333′ on a circumferential outer surface thereof. The general construction and function of the fixture 3′, other than the cortical bone coupling part 33′, are equal to those of the fixture 3 according to the embodiment of FIG. 5. Therefore, hereinafter, only the large cortical screw thread 333′ and the small cortical screw thread 331′ will be explained, after the process of manufacturing the fixture 3′ is briefly described.

The large screw threads 333′ and 343′ are formed on the outer surface of the fixture using a single bit tool. Here, in the process of forming the large cancellous screw. thread 343′, the insertion depth of the bit tool is maintained constant, such that the root diameter d1′ and the ridge width H1′ of the large cancellous screw thread 343′ are constant throughout the cancellous bone coupling part. Meanwhile, in the process of forming the large cortical screw thread 333′, the bit tool is retracted to a predetermined ratio as the bit tool works from the lower end of the cortical bone coupling part to the uppermost part 31′. Thus, the root diameter d5′ and the ridge width H2′ of the large cortical screw thread 333′ are increased from the lower end of the cortical bone coupling part to the uppermost part 31′. Therefore, because the large cancellous screw thread 343′ and the large cortical screw thread 333′ are machined using the same bit tool, their pitches p1′ and p3′ are equal. However, the ridge width H2′ of the large cortical screw thread 333′ is greater than the ridge width H1′ of the large cancellous screw thread 343′ and is increased from the lower end thereof to the upper end thereof. As such, due to the difference in ridge width, a larger number of small screw threads can be formed on the large cortical screw thread, compared to the small cancellous screw thread.

The large cortical screw thread 333′ extends from the large cancellous screw thread 343′ and has an outer diameter d6′, a root diameter d5′ and a pitch p3′. The large cortical screw thread 333′ has a trapezoidal shape and is provided with small cortical screw threads 331′ in the ridge thereof. The pitch p3′ and number of the large cortical screw thread 333′ are equal to the pitch p1′ and number of the large cancellous screw thread 343′, so that the lead of the large cortical screw thread 333′ is equal to that of the large cancellous screw thread 343′.

The root diameter d5′ of the large cortical screw thread 333′ is gradually increased from the lower end to the upper end of the fixture 3′ and becomes equal to the root diameter d7′ of the small cortical screw thread 331′. The outer diameter d6′ of the large cortical screw thread 333′ is equal to the outer diameter d3′ of the large cancellous screw thread 343′. Therefore, in the large cortical screw thread 333′, the difference between the outer diameter d6′ and the root diameter d5′ is reduced from the lower end thereof to the upper end thereof. Thus, compared to the conventional fixture 1, stress concentration around the cortical bone coupling part 33′ is reduced. Furthermore, the root diameter d5′ of the large cortical screw thread is gradually increased from the lower end thereof to the upper end thereof. Accordingly, thanks to the resultant wedging effect, the fixture of this embodiment can be more reliably fastened to the bone tissue after the implantation of the fixture has been completed.

The several small cortical screw threads 331′ are formed on the ridge of the large cortical screw thread 333′. Each small cortical screw thread 331′ has an outer diameter d8′, a root diameter d7′ and a pitch d4′. Preferably, the number of small cortical screw threads 331′ is greater than the number of small cancellous screw threads 341′ formed on the circumferential outer surface of the cancellous bone coupling part 34′, such that stress relative to the cortical bone is distributed to the cancellous bone. In this embodiment, although three small cortical screw threads 331′ are formed on the large cortical screw thread 333′, the number of small cortical screw threads 331′ may be variously changed according to the intended purpose, so long as the number of small cortical screw threads 331′ is greater than the number of small cancellous screw threads.

Furthermore, in the fixture 3′ of this embodiment, the ridge width H2′ of the large cortical screw thread is greater than the ridge width H1′ of the large cancellous screw thread and is increased from the lower end thereof to the upper end thereof. Therefore, although a number of small cortical screw threads that is greater than the number of small cancellous screw threads is machined in the ridge of the large cortical screw thread, the ridge width of each small cortical screw thread is prevented from being excessively narrowed. As well, because the contact surface of the fixture with the cortical bone is increased, the fixture can be more reliably fastened to the bone.

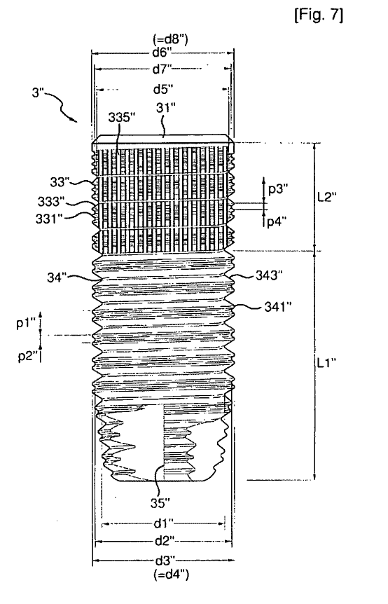

Meanwhile, as shown in FIG. 7, a fixture 3″ according to still another embodiment includes an uppermost part 31″, a cortical bone coupling part 33″, a cancellous bone coupling part 34″, a cutting edge 35″, an adaptor seating hole, and a screw locking hole. Furthermore, a large cancellous screw thread 343″ is formed on the circumferential outer surface of the cancerous bone coupling part 34″, and small cancellous screw threads 341″ are formed on the ridge of the large cancellous screw thread 343″, in the same manner as those of the embodiment shown in FIG. 6. As well, a large cortical screw thread 333″ and a small cortical screw thread 331″ are formed on the circumferential outer surface of the cortical bone coupling part 33″. The general construction and function of the fixture 3″, other than longitudinal grooves 335″ formed in the circumferential outer surface of the cortical bone coupling part 33″, are equal to those of the fixture 3′ according to the former embodiment, therefore, only the longitudinal grooves 335″ will be explained herein below.

The longitudinal grooves 335″ are formed throughout the entire circumferential outer surface of the cortical bone coupling part 34″, on which the small cortical screw threads 331″ are formed. The longitudinal grooves 335″ are spaced apart from each other at regular intervals. The longitudinal grooves 335″ cross the small cortical screw threads 331″, so that a lattice shape is formed on the circumferential outer surface of the cortical bone coupling part 34″. Therefore, due to the longitudinal grooves 335″, stress related to the cortical bone coupling part 34″ is distributed to the cancellous bone coupling part 35″, thus preventing stress concentration in the cortical bone.

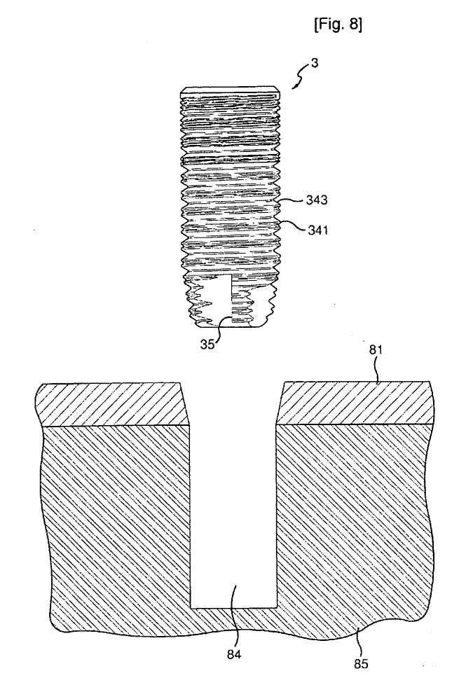

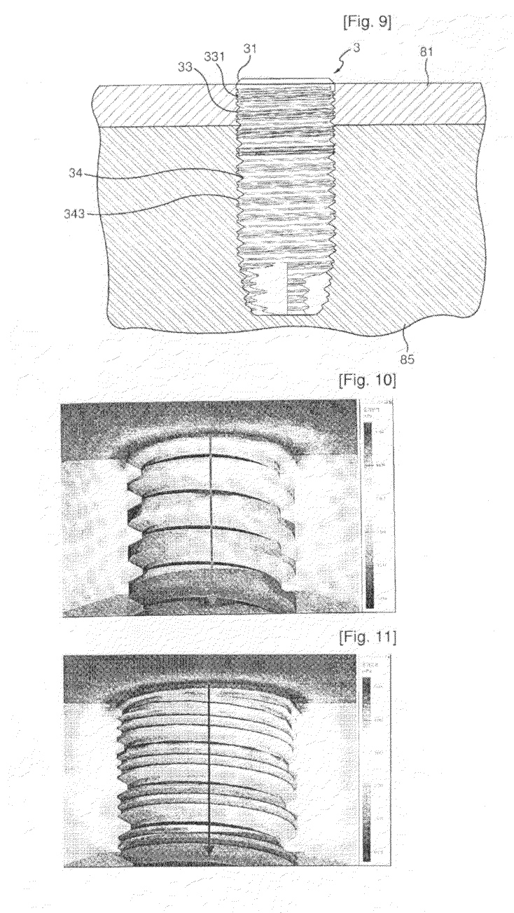

Hereinafter, a process of implanting the fixture 3′ will be described with reference to FIGS. 8 and 9.

As shown in FIG. 8, an insertion hole 84, in which the fixture 3′ of the present invention is to be implanted, is formed in the bone using a drill (not shown) to a depth corresponding to the length of the fixture 3′. Here, the insertion hole 84 has a diameter slightly less than the root diameter d1′ of the large cancellous screw thread of the fixture 3′. Therefore, when the fixture 3′ is tightened into the insertion hole 84, the cutting edge 35′ taps the circumferential inner surface of the insertion hole 84, thus forming an internal thread on the circumferential inner surface. The fixture 3′ is screwed into the internal thread of the insertion hole 84 and is thus fixed to the bone.

FIG. 9 is a sectional view showing the fixture 3′ implanted in the bone tissue. Referring to FIG. 9, some of the cortical bone coupling part 33′ is placed in the cortical bone 81, and the cancellous bone coupling part 34′ is placed in the cancellous bone 83. The uppermost part 31′ protrudes outside the cortical bone 81. The fixture 3′ of the present invention has the large screw threads 333′ and 343′ on the circumferential outer surface thereof and has the small cortical screw threads and the small cancellous screw threads formed on the ridges of the large screw threads. Therefore, the contact surface of the fixture with the alveolar bone is increased. As well, thanks to the wedging effect of the large cortical screw thread having the root diameter, which enlarges from the lower end thereof to the upper end thereof reliable coupling force of the fixture to the alveolar bone is ensured. Furthermore, the large cancellous screw thread 343′, which is disposed in the cancellous bone 83 being relatively smooth bone tissue, provides the fixing force to the fixture 3′, and the stress concentration around the large cancellous screw thread 343′ is distributed by the small cancellous screw threads 341′. In addition, the number of small cortical screw threads of the cortical bone coupling part 33′ is greater than that of the large cancellous screw thread 343′, and the root diameter d5′ of the cortical bone coupling part 33′ is greater than the root diameter d1′ of the cancellous bone coupling part. Therefore, stress applied to the cortical bone coupling part 33′ is distributed to the cancellous bone coupling part 34′, so that the stress distribution of the entire fixture becomes even.

Hereinafter, the effect of the present invention will be described through the following test.

### Test 1: Estimation of Stress Applied To Screw Threads of the Fixture Using A Finite Element Method (FEM)

### Test Conditions

Fixture of the present invention of FIG. 6 and the conventional fixture of FIG. 1 were implanted in bone tissue comprising cancellous bone and cortical bone, and abutments were coupled to the respective fixtures. Thereafter, a vertical stress of 100N was applied to each fixture in a state in which both sides of the bone were fixed. At this time, a linear FEM analysis of the screw threads of each fixture was conducted using the 3G program of Plasso Tech Ltd. Here, an auto mesh function was used for meshing of the model, and stress distribution applied to the cortical bone coupling part was analyzed. The materials and physical properties of the cortical bone, the cancellous bone and the fixture used in the test are described in the following Table 1.

TABLE 1

| | | Elastic co- | |
|---|---|---|---|
| Parts | Material | efficient (GPa) | Poisson's ratio |
| fixture | titanium | 120 | 0.3 |
| cortical bone | bone | 13.7 | 0.33 |
| cancellous bone | bone | 1.37 | 0.33 |

### Test Result

Stress distribution of the conventional fixture is shown in FIG. 10, and stress distribution of the fixture of the present invention is shown in FIG. 11. Referring to FIG. 10, in the conventional fixture, stress is concentrated in the end of the screw thread of the fixture. However, in the present invention, as shown in FIG. 11, thanks to the small screw threads formed on the large screw thread, the stress related to the large screw thread is distributed to the small screw threads. Therefore, it will be appreciated that the fixture of the present invention prevents bone resorption and promotes osseointegration due to the mitigated stress concentration compared to the conventional fixture.

### INDUSTRIAL APPLICABILITY

Although the preferred embodiments of the present invention have been disclosed for illustrative purposes t must be appreciated that various modifications, additions and substitutions realizing the technical idea of the invention fall within the bounds of the present invention. For example, the fixture of the present invention may be used for orthopedics as well as for dental implants. Furthermore, in the above-mentioned embodiments, although internal type fixtures have been illustrated, the present invention may be applied to external type fixtures.

1. A fixture, having a screw shape, to be implanted in bone tissue, the fixture comprising an uppermost part protruding outside the bone tissue when implanted in the bone tissue and a body part placed in the bone tissue, the body part having a cortical bone coupling part installed in cortical bone and a cancellous bone coupling part installed in cancellous bone, wherein the cancellous bone coupling part comprises a large cancellous screw thread formed on a circumferential outer surface of the cancellous bone coupling part and a small cancellous screw thread formed on a ridge of the large cancellous screw thread, and the cortical bone coupling part comprises a small cortical screw thread having a pitch, a root diameter and an outer diameter almost equal to a pitch, a root diameter and an outer diameter of the small cancellous screw thread.

2. A fixture, having a screw shape, to be implanted in bone tissue, the fixture comprising an uppermost part protruding outside the bone tissue when implanted in the bone tissue and a body part placed in the bone tissue, the body part comprising a cortical bone coupling part installed in cortical bone and a cancellous bone coupling part installed in cancellous bone, wherein the cancellous bone coupling part comprises a large cancellous screw thread formed on a circumferential outer surface of the cancellous bone coupling part and a small cancellous screw thread formed on a ridge of the large cancel bus screw thread, and the cortical bone coupling part comprises a large cortical screw thread extending from the large cancellous screw thread and a small cortical screw thread formed on a ridge of the large cortical screw thread, wherein the number of small cortical screw threads is greater than the number of small cancellous screw threads.

3. The fixture according to claim 2, wherein the large cortical screw thread has a lead equal to a lead of the large cancellous screw thread,

4. The fixture according to claim 3, wherein a root diameter of the large cortical screw thread is increased from a lower end thereof to an upper end thereof and is equal to a root diameter of the small cortical screw thread at the upper end thereof.

5. The fixture according to claim 4, wherein a root diameter of the large cancellous screw thread is constant throughout the cancellous bone coupling part.

6. The fixture according to claim 1, further comprising:
a plurality of longitudinal grooves formed in a circumferential outer surface of the cortical bone coupling part.

7. The fixture according to claim 2, further comprising:
a plurality of longitudinal grooves formed in a circumferential outer surface of the cortical bone coupling part.

8. The fixture according to claim 3, further comprising:
a plurality of longitudinal grooves formed in a circumferential outer surface of the cortical bone coupling part.

9. The fixture according to claim 4, further comprising:
a plurality of longitudinal grooves formed in a circumferential outer surface of the cortical bone coupling part.

10. The fixture according to claim 5, further comprising:
a plurality of longitudinal grooves formed in a circumferential outer surface of the cortical bone coupling part.
